(19) United States
(12) Patent Application Publication
(10) Pub. No.: US 2016/0250139 A1
(43) Pub. Date: Sep. 1, 2016

(54) HAIR TREATMENT AGENTS HAVING SUBSTITUTED SILICONE(S)

(71) Applicant: Henkel AG & Co. KGaA, Duesseldorf (DE)

(72) Inventors: Erik Schulze zur Wiesche, Hamburg (DE); Rene Krohn, Norderstedt (DE)

(73) Assignee: Henkel AG & Co. KGaA, Duesseldorf (DE)

(21) Appl. No.: 15/151,675

(22) Filed: May 11, 2016

Related U.S. Application Data
(63) Continuation of application No. PCT/EP2014/073633, filed on Nov. 4, 2014.

(30) Foreign Application Priority Data
Dec. 5, 2013 (DE) 10 2013 224 991.8

(51) Int. Cl. A61K 8/898; A61K 8/86; A61K 8/33; A61K 8/39; A61K 8/81; A61Q 5/00

(52) U.S. Cl. CPC A61K 8/898; A61K 8/8152; A61Q 5/00; A61K 8/33; A61K 8/39; A61K 8/86; A61K 2800/594

(57) ABSTRACT

The invention relates to a hair treatment agents, including—in each case with respect to the weight of the hair treatment agents—0.1 to 20 wt % of at least one silicone, which has groups of formula (I) as defined herein; 0.1 to 20 wt % of at least one copolymer, which has monomer units of formulas (V) and (VI) as defined herein; and 0.1 to 10 wt % of (RS)-2-cyano-3,3-diphenylacrylic acid-2-ethylhexylester. The hair treatment agents have an improved protective effect against UV radiation and give hair treated with said hair treatment agents better combability, shine, elasticity, brittleness, and maximum tear resistance.

### FIELD OF THE INVENTION

The present invention generally relates to hair treatment agents including specially substituted silicone(s) and also to the use of these agents to clean and/or care for hair.

### BACKGROUND OF THE INVENTION

Care products for keratin fibers influence the natural structure and the properties of the hair. Following treatments of this type, the wet and dry combability of the hair, the hold, and the volume of the hair, for example, can be optimized, or the hair can be protected against increased splitting. It has therefore long been usual to subject the hair to a special post-treatment. Here, the hair is treated with special active substances, for example quaternary ammonium salts or special polymers, usually in the form of a rinse. As a result of this treatment, depending on the formulation, the combability, the hold, and the volume of the hair are improved, and the splitting rate is reduced.

By way of example, due to the intensive use of tanning salons or extensive physical leisure activities, the structure of hair is more heavily compromised in a lasting manner as a result of UV light. This damage manifests itself on the hair for example in the form of a loss of elasticity. It is therefore not surprising that the proportion of consumers with sensitive, brittle and irritated skin having poor elasticity and also with hair that is of poorer quality in terms of combability, shine, elasticity, brittleness and maximum tear resistance is significantly increasing.

A further problem lies in colored hair. If the dyes formed or used directly during the course of the coloring have considerably different fastness properties (for example UV stability, perspiration fastness, washing fastness, etc.), this may result over time in a noticeable and therefore undesirable color change. This phenomenon occurs in a more pronounced manner when the hairstyle, hair or hair zones has/have different degrees of damage. One example is long hair, in which case the hair ends exposed over a long time to all possible environmental influences are generally damaged much more heavily than the relatively freshly grown hair zones.

There is thus a need to provide care products that include a high protective effect against the fading of natural or dyed hair color and against damage to the hair structure as a result of UV radiation.

It has therefore long been conventional, both in skin and hair cosmetics, to incorporate UV filters into the agents. According to European law, UV filters are defined in terms of their purpose. They are therefore “substances, which are intended exclusively or primarily to protect the skin against specific UV radiation by absorption, reflection or scattering of specific UV radiation”. This definition is attributed to all UV filters, regardless of whether they are organic or inorganic, so-called chemical or physical, natural or artificial UV filters. Only the UV filters that are listed in the Cosmetics Regulation may be used. Since the individual substances generally do not offer protection across the entire UV spectrum, a number of substances are usually combined. In addition, synergistic effects usually result from the combination of UV filters.

Whereas the formulation of UV protection in skincare products is widely elaborated, less complex systems are used in hair care products for “dead” hair material. In addition, the amounts in which filter substances can be incorporated are also usually smaller, partly due to formulation reasons and partly due to cost reasons. So as to nevertheless provide effective UV protection, there is a need to improve the protective effect of known and economical UV filters and to provide the corresponding agents additionally with positive effects with respect to combability, shine, elasticity, brittleness, and maximum tear resistance of the hair treated therewith.

Furthermore, other desirable features and characteristics of the present invention will become apparent from the subsequent detailed description of the invention and the appended claims, taken in conjunction with this background of the invention.

### BRIEF SUMMARY OF THE INVENTION

A hair treatment agent includes—in each case with respect to the weight of the hair treatment agent—

a) 0.1 to 20 wt % of at least one silicone, which has groups of formula (I)

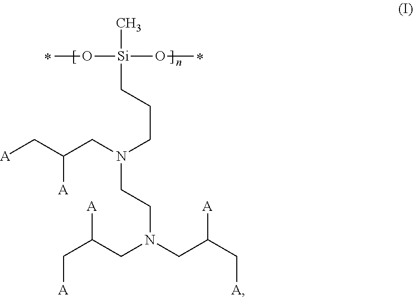

wherein n stands for values from 1 to 1000 and A stands for a group

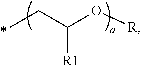

wherein R1 stands for —H or —CH₃ and R stands for a straight-chained or branched alkyl group having 8 to 24 C atoms and a stands for an integer from 1 to 20;
b) 0.1 to 20 wt % of at least one copolymer, which has monomer units of formulas (V) and (VI)

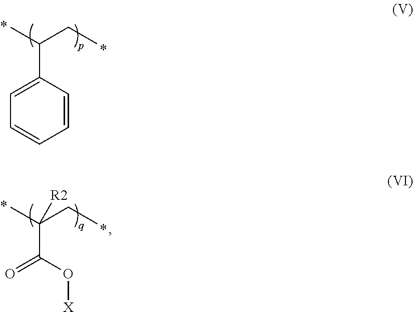

in which R2 stands for —H or —CH₃ and X stands for —H, an alkali metal cation, or a straight-chained or branched alkyl group having 1 to 24 C atoms; and
c) 0.1 to 10 wt % of (RS)-2-cyano-3,3-diphenylacrylic acid-2-ethylhexylester.

Silicones which have groups of formula (I) are used for reducing damage to the hair structure caused by external influences, in particular by UV radiation

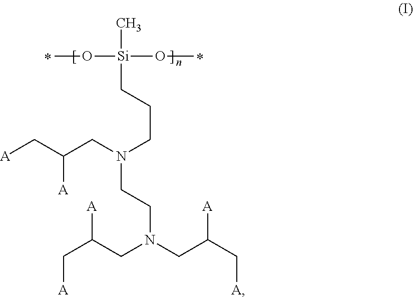

wherein n stands for values from 1 to 1000 and A stands for a group

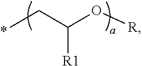

wherein R1 stands for —H or —CH₃ and R stands for a straight-chained or branched alkyl group having 8 to 24 C atoms and a stands for an integer from 1 to 20.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that octocrylene-containing agents can be significantly improved in respect to the above-mentioned series of tasks when certain acrylate polymers and substituted silicones are used.

A first subject matter of the present invention is a hair treatment agent, including
in each case with respect to the weight of the hair treatment agent—
   a) 0.1 to 20 wt % of at least one silicone, which has groups of formula (I)

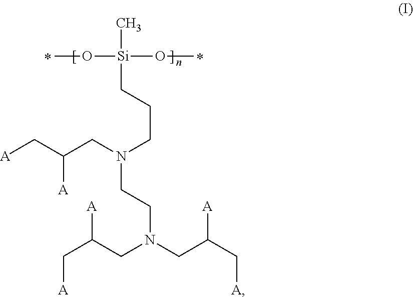

in which
n stands for values from 1 to 1000 and
A stands for a group

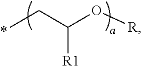

in which R1 stands for —H or —CH₃ and R stands for a straight-chained or branched alkyl group having 8 to 24 C atoms and a stands for an integer from 1 to 20;
b) 0.1 to 20 wt % of at least one copolymer, which has monomer units of formulas (V) and (VI)

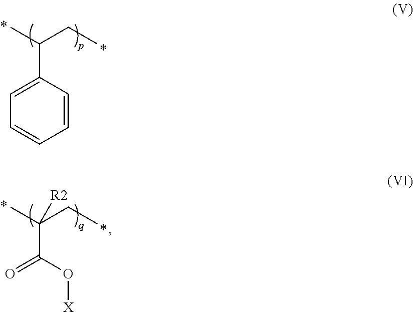

in which R2 stands for —H or —CH₃ and X stands for —H, an alkali metal cation, or a straight-chained or branched alkyl group having 1 to 24 C atoms;
c) 0.1 to 10 wt % of (RS)-2-cyano-3,3-diphenylacrylic acid-2-ethylhexylester.

Hair treatment agents in the sense of the present invention are, for example, hair shampoos, hair conditioners, conditioning shampoos, hairsprays, hair rinses, hair treatments, hair packs, hair tonics, permanent wave fixing solutions, coloring shampoos, coloring agents, hair fixers, hair setting agents, hair styling preparations, blow-dry lotions, foam fixers, hair gels, hair waxes, or combinations thereof. In view of the fact that men often avoid the application of a number of different agents and/or a number of application steps, agents according to the invention are preferably agents that can be readily applied by men. Preferred agents according to the invention are therefore shampoos, conditioning agents or hair tonics.

The compositions of the invention provide the keratin fibers treated therewith with improved properties, which are maintained even under intensive UV irradiation (for example lightness, softness, detangling ability, natural feeling, and airy hairstyle, brightness), and in addition the effects are persistent and lasting. In particular, these effects are resistant to many shampoos.

The agents according to the invention include, as first essential ingredient, 0.1 to 20 wt % of at least one silicone, which comprises groups of formula (I), In formula (I) n stands for values from 1 to 1000, wherein preferred values are 1 to 40, preferably 1 to 30, more preferably 1 to 20, and in particular the values 2, 3, 4, 5, 6, 7, 8, 9 or 10.

In formula (I) A stands for a group

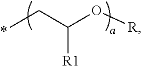

in which R1 stands for —H or —CH₃ and R stands for a straight-chained or branched alkyl group having 8 to 24 C atoms and a stands for an integer from 1 to 20.

Particularly preferably R1=—H.

Particularly good UV protection is attained when the group R stands for
—(CH₂)ₖ—CH₃ with k=7, 8, 9, 10, 11, 12, 13, 14, 15, 16 or 17
—CH(CH₂CH₃)—(CH₂)₅.

In accordance with the invention, particularly preferred hair treatment agents are characterized in that they include 0.1 to 20 wt %, preferably 0.25 to 15 wt %, more preferably 0.5 to 10 wt %, even more preferably 0.75 to 7.5 wt %, and in particular 1 to 5 wt % of at least one silicone, which comprises groups of formula (Ia)

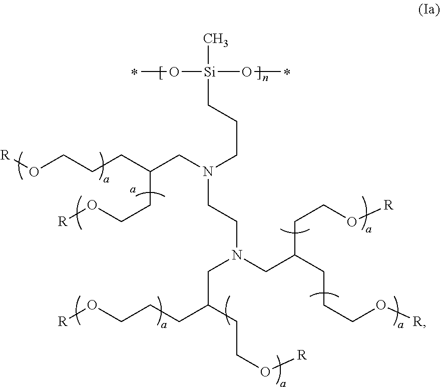

in which R is selected from
—(CH₂)ₖ—CH₃ with k=7, 8, 9, 10, 11, 12, 13, 14, 15, 16 or 17
—CH(CH₂CH₃)—(CH₂)₅

and a stands for an integer from 1 to 20.

The silicones used in accordance with the invention can consist completely of groups of formula (I) or (Ia), wherein —CH₃, —O—CH₃ or —OH are preferred as end groups.

However, it is preferable for the silicones used in accordance with the invention to include further groups, in particular those of formula

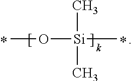

Co silicones formed from units of the above-mentioned formula and units of formula (I) or (Ia) can also have —CH₃, —O—CH₃ or —OH as end groups. The units can be present in such Co silicones in blocks or randomly distributed.

Particularly preferred hair treatment agents according to the invention are characterized in that they include 0.1 to 20 wt %, preferably 0.25 to 15 wt %, more preferably 0.5 to 10 wt %, even more preferably 0.75 to 7.5 wt %, and in particular 1 to 5 wt % of at least one silicone of formula (II):

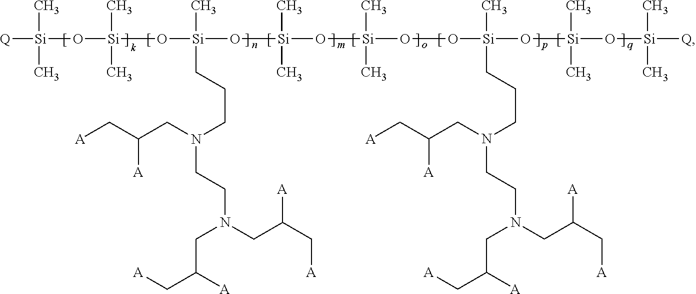

in which
A stands for a group

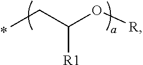

in which R1 stands for —H or —CH₃ and R stands for a straight-chained or branched alkyl group having 8 to 24 C atoms and a stands for an integer from 1 to 20;

Q stands for —OH or —CH₃ or —OCH₃;

the indices k, n, m, o, p and q stand for integers from 0 to 1000, provided that
n+p>0 and k+m+o+q>0
   wherein the units with the indices k, n, m, o, p, q can be present in the molecule as a block or randomly distributed.

In the case of the silicones of formula (II) as well, A stands for a group

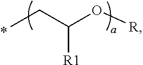

in which R1 stands for —H or —CH₃ and R stands for a straight-chained or branched alkyl group having 8 to 24 C atoms and a stands for an integer from 1 to 20;

Particularly preferably R1=H.

Particularly good UV protection is attained when the group R stands for
—(CH₂)ₖ—CH₃ with k=7, 8, 9, 10, 11, 12, 13, 14, 15, 16 or 17
—CH(CH₂CH₃)—(CH₂)₅.

Further preferred hair treatment agents according to the invention are therefore characterized in that they include 0.1 to 20 wt %, preferably 0.25 to 15 wt %, more preferably 0.5 to 10 wt %, even more preferably 0.75 to 7.5 wt %, and in particular 1 to 5 wt % of at least one silicone of formula (III):

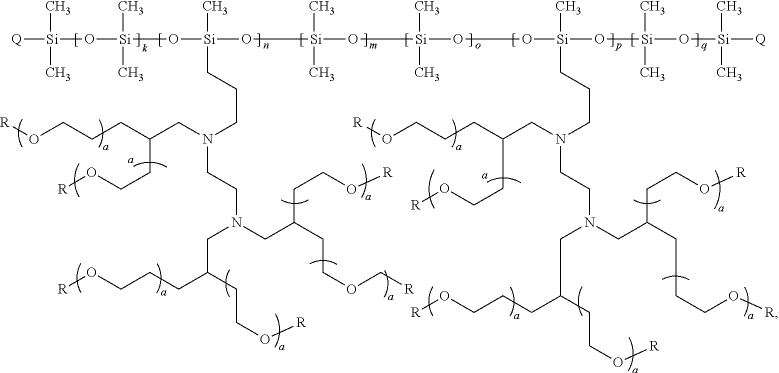

in which

R is selected from
—(CH₂)ₖ—CH₃ with k=7, 8, 9, 10, 11, 12, 13, 14, 15, 16 or 17
—CH(CH₂CH₃)—(CH₂)₅

a stands for an integer from 1 to 20;

Q stands for —OH or —CH₃ or —OCH₃;

the indices k, n, m, o, p and q stand for integers from 0 to 1000, provided that
n+p>0 and k+m+o+q>0
   wherein the units with the indices k, n, m, o, p, q can be present in the molecule as a block or randomly distributed.

The index a most preferably stands for the number 9, and R is a straight-chained alkyl group having 13 carbon atoms. Particularly preferred hair treatment agents are therefore characterized in that they include 0.1 to 20 wt %, preferably 0.25 to 15 wt %, more preferably 0.5 to 10 wt %, even more preferably 0.75 to 7.5 wt %, and in particular 1 to 5 wt % of at least one silicone of formula (IV):

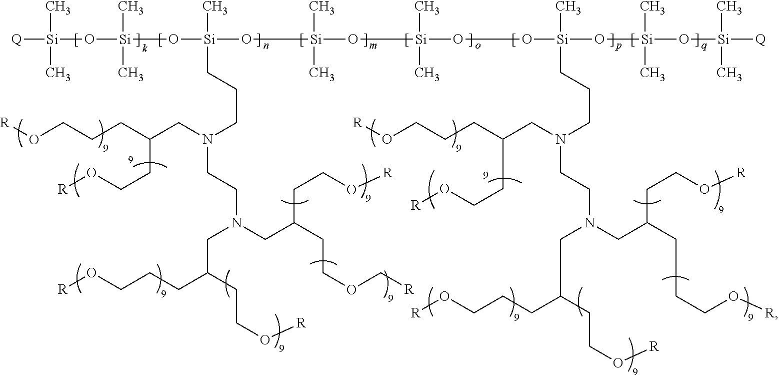

in which

R stands for —(CH₂)₁₂—CH₃ and Q stands for —OH or —OCH₃;

Q stands for —OH or —CH₃ or —OCH₃;

the indices k, n, m, o, p and q stand for integers from 0 to 1000, provided that
n+p>0 and k+m+o+q>0
   wherein the units with the indices k, n, m, o, p, q can be present in the molecule as a block or randomly distributed.

In formulas (II), (III), and (IV), Q stands for —OH or —CH₃ or —OCH₃. In all three formulas the two Q must not be identical, rather the termination at one end can be different from that at the other end, wherein the following combinations are preferred:

| “left” end group Q | “right end group” |
|---|---|
| —OH | —OH |
| —CH₃ | —OH |
| —OCH₃ | —OH |
| —OH | —CH₃ |
| —CH₃ | —CH₃ |
| —OCH₃ | —CH₃ |
| —OH | —OCH₃ |
| —CH₃ | —OCH₃ |
| —OCH₃ | —OCH₃. |

The two groups at each of the chain ends are particularly preferably identical, and Q particularly preferably stands for —OH or —OCH₃. The silicones of formulas (IIa), (IIb), (IIIa), (IIIb), (IVa) and (IVb) are particularly preferred embodiments:

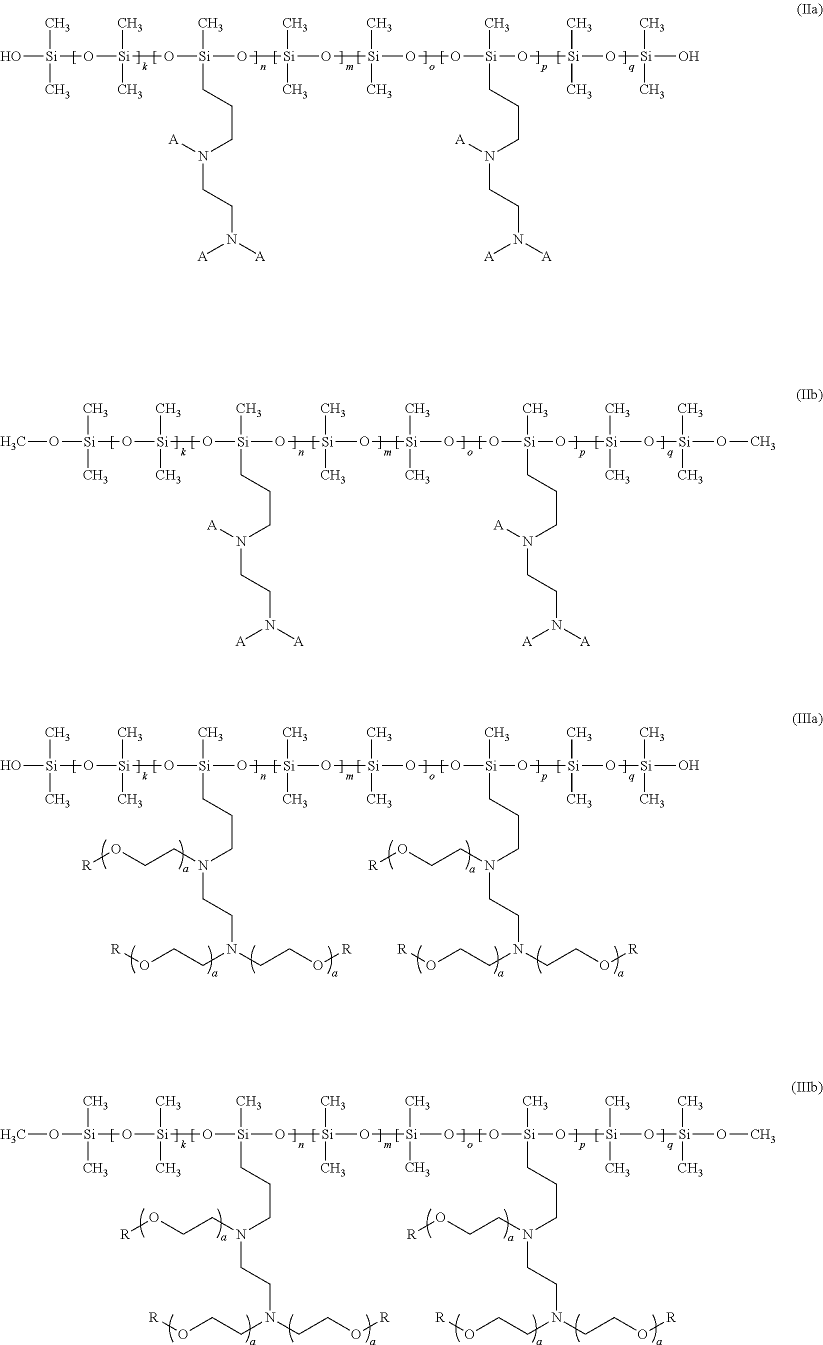

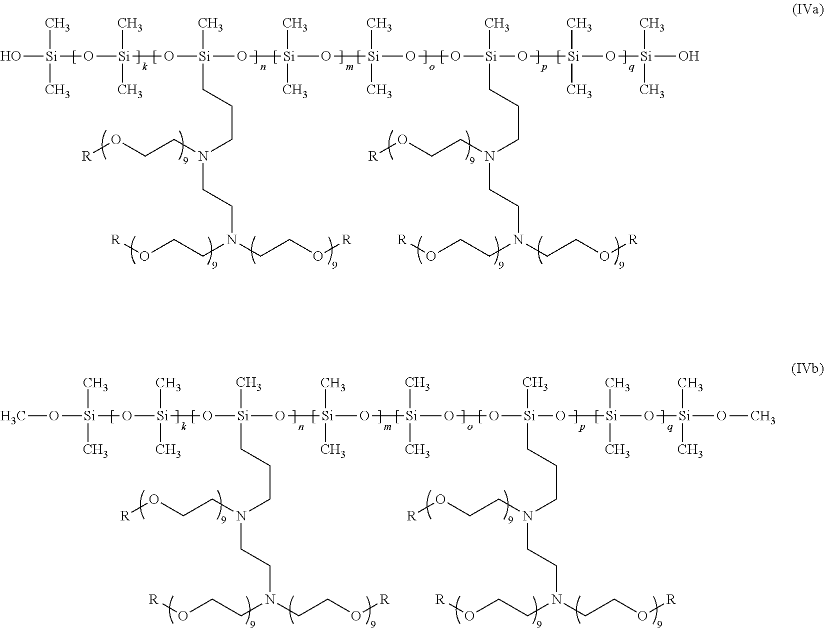

The mean molecular weights of the silicones a) included in the agents according to the invention is preferably from 2,000 to 300,000 and even more preferably from 5,000 to 200,000, in particular 10,000 to 150,000 Dalton. Hair treatment agents in which the weight-average molar mass of the silicone of formula (IV) included therein lies in the range from 10,000 to 150,000 gmol⁻¹, preferably in the range from 50,000 to 130,000 gmol⁻¹, are preferred.

The mean molecular weights of amino-substituted silicones can be measured for example by gel permeation chromatography (GPC) at room temperature in polystyrene. Styragel columns μ can be selected as columns, THF can be selected as eluent, and a flow rate of 1 ml/min can be selected. The detection is performed preferably by means of refractometry and UV meters.

As a further constituent, the agents according to the invention include 0.1 to 20 wt % of at least one copolymer, which has monomer units of formulas (V) and (VI)

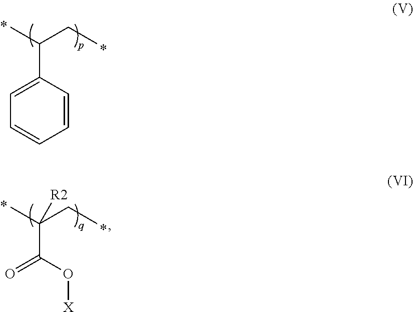

in which R2 stands for —H or —CH₃ and X stands for —H, an alkali metal cation, or a straight-chained or branched alkyl group having 1 to 24 C atoms.

The copolymers can consist only of monomers of formulas (V) and (VI), but can additionally also include further monomer building blocks. In view of the protection against damage to the hair structure, it has proven to be preferable when at least 50 wt %, preferably at least 60 wt %, more preferably at least 70 wt %, even more preferably at least 80 wt %, particularly preferably at least 90 wt %, and in particular at least 99 wt % of the copolymers consist of monomers of the formulas (V) and (VI).

The monomer to be allocated to the structural unit (V) is styrene; the structural unit (VI) can be derived from acrylic acid (R2=—H, X=—H), methacrylic acid (R2=—CH₃, X=—H), salts thereof (X=Na₊, K₊, etc.) and/or esters thereof (X=alkyl). The copolymers can include a single type of monomer unit (VI) or a plurality of different monomer units (VI, VI′, VI″, etc.). An example for the first embodiment would be copolymers of styrene and acrylic acid, and an example for the second embodiment would be copolymers of styrene, acrylic acid and methacrylate.

Among the copolymers that include exclusively monomer units of formulas (V) and (VI), the following copolymers are preferred:
copolymers of styrene and acrylic acid
copolymers of styrene and methacrylic acid
copolymers of styrene and sodium acrylate
copolymers of styrene and sodium methacrylate
copolymers of styrene and potassium acrylate
copolymers of styrene and potassium methacrylate
copolymers of styrene and methyl acrylate
copolymers of styrene and methyl methacrylate
copolymers of styrene and ethyl acrylate
copolymers of styrene and ethyl methacrylate
copolymers of styrene and propyl acrylate
copolymers of styrene and propyl methacrylate
copolymers of styrene and isopropyl acrylate
copolymers of styrene and isopropyl methacrylate
copolymers of styrene and acrylic acid (n-butyl) ester
copolymers of styrene and methacrylic acid (n-butyl) ester
copolymers of styrene and acrylic acid and methacrylic acid
copolymers of styrene and acrylic acid and sodium acrylate
copolymers of styrene and acrylic acid and sodium methacrylate
copolymers of styrene and acrylic acid and methyl acrylate
copolymers of styrene and acrylic acid and methyl methacrylate
copolymers of styrene and acrylic acid and ethyl acrylate
copolymers of styrene and acrylic acid and ethyl methacrylate
copolymers of styrene and acrylic acid ester and acrylic acid (n-butyl) ester
copolymers of styrene and acrylic acid and methacrylic acid (n-butyl) ester
copolymers of styrene and methacrylic acid and sodium acrylate
copolymers of styrene and methacrylic acid and sodium methacrylate
copolymers of styrene and methacrylic acid and methyl acrylate
copolymers of styrene and methacrylic acid and ethyl methacrylate
copolymers of styrene and methacrylic acid and ethyl acrylate
copolymers of styrene and methacrylic acid and ethyl methacrylate
copolymers of styrene and methacrylic acid ester and acrylic acid (n-butyl) ester
copolymers of styrene and methacrylic acid and methacrylic acid (n-butyl) ester
copolymers of styrene and acrylic acid and methacrylic acid and sodium acrylate
copolymers of styrene and acrylic acid and methacrylic acid and sodium methacrylate
copolymers of styrene and acrylic acid and methacrylic acid and methyl acrylate
copolymers of styrene and acrylic acid and methacrylic acid and methyl methacrylate
copolymers of styrene and acrylic acid and methacrylic acid and ethyl acrylate
copolymers of styrene and acrylic acid and methacrylic acid and ethyl methacrylate
copolymers of styrene and acrylic acid and methacrylic acid and acrylic acid (n-butyl) ester
copolymers of styrene and acrylic acid and methacrylic acid and methacrylic acid (n-butyl) ester).

Particularly preferred hair treatment agents are therefore characterized in that they include 0.5 to 17.5 wt %, preferably 1 to 15 wt %, more preferably 1.5 to 12.5, particularly preferably 2 to 10, and in particular 3 to 8 wt % of at least one copolymer, which has monomer units of the formulas (V) and (VIa)

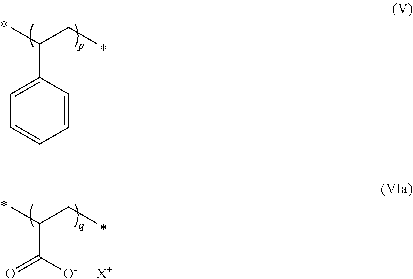

in which X stands for H⁻ or Na⁺.

As further constituent, the agents according to the invention include 0.1 to 10 wt % of (RS)-2-cyano-3,3-diphenylacrylic acid-2-ethylhexylester. Particularly preferred hair treatment agents according to the invention are characterized in that they include 0.25 to 9 wt %, preferably 0.5 to 8 wt %, more preferably 0.75 to 7 wt %, particularly preferably 1 to 6 wt %, and in particular 2.5 to 5 wt % of (RS)-2-cyano-3,3-diphenylacrylic acid-2-ethylhexyl ester.

It has been found to be preferable when ingredients a), b) and c) are used in certain ratios by weight to one another. In this way, the UV protection is optimally intensified. Hair treatment agents that are preferred in accordance with the invention are characterized in that the ratio by weight of ingredients b) to a) lies in the range from 1:50 to 50:1, preferably in the range from 1:10 to 25:1, more preferably in the range from 1:1 to 5:1, particularly preferably in the range from 1.5:1 to 4:1, and in particular in the range from 1.75:1 to 2.5:1.

More preferred hair treatment agents according to the invention are characterized in that the ratio by weight of ingredients a) to c) lies in the range from 1:50 to 50:1, preferably in the range from 1:10 to 25:1, more preferably in the range from 1:5 to 5:1, particularly preferably in the range from 1.5:1 to 4:1, and in particular in the range from 1.1:1 to 1:1.1.

Depending on their intended purpose, the agents according to the invention include further essential ingredients. Cleaning or caring compositions, such as shampoos or conditioners, include at least one surfactant, wherein surface-active substances are referred to as surfactants or emulsifiers depending on the field of application and are selected from anionic, cationic, zwitterionic, ampholytic, and non-ionic surfactants and emulsifiers.

Hair treatment agents that are preferred in accordance with the invention are characterized in that they include—with respect to the weight of said hair treatment agents—0.5 to 70 wt %, preferably 1 to 60 wt %, and in particular 5 to 25 wt % of anionic and/or non-ionic and/or cationic and/or amphoteric surfactant(s).

All anionic surface-active substances suitable for use on the human body are suitable as anionic surfactants and emulsifiers for the compositions according to the invention. These are characterized by an anionic group conveying water solubility, such as a carboxylate, sulfate, sulfonate or phosphate group and a lipophilic alkyl group having approximately 8 to 30 C atoms. In addition, glycol or polygycol ether groups, ester, ether and amide groups, and hydroxyl groups can be included in the molecule.

Surface-active compounds that carry at least one quaternary ammonium group and at least one —COO⁽⁻⁾ or —SO₃⁽⁻⁾ group in the molecule are designated as zwitterionic surfactants and molecules. Particularly suitable zwitterionic surfactants and emulsifiers are the so-called betaines, such as N-alkyl-N,N-dimethyl ammonium glycinates, for example coco-alkyl dimethyl ammonium glycinate, N-acyl-aminopropyl-N,N-dimethyl ammonium glycinates, for example coco-acyl aminopropyl dimethyl ammonium glycinate, and 2-alkyl-3-carboxymethyl-3-hydroxyethyl-imidazolines, each having 8 to 18 C atoms in the alkyl or acyl group, as well as coco-acyl aminoethyl hydroxyl ethyl carboxymethyl glycinate. A preferred zwitterionic surfactant is the fatty acid amide derivative known under the INCI name cocamidopropyl betaine.

Further preferred anionic surfactants are alkyl sulfates, alkyl polyglycol ether sulfates and ether carboxylic acid salts having 10 to 18 C atoms in the alkyl group and up to 12 glycol ether groups in the molecule and sulfosuccinic monoesters and dialkyl esters having 8 to 18 C atoms in the alkyl group and sulfosuccinic acid monoalkyl polyoxyethyl esters having 8 to 18 C atoms in the alkyl group and 1 to 6 oxyethyl groups.

Particularly preferred anionic surfactants are the alkali or ammonium salts of lauryl ether sulfate with a degree of ethoxylation from 2 to 4 EO.

Particularly preferred cosmetic agents according to the invention are characterized in that they—with respect to their weight—include 0.1 to 20 wt %, preferably 0.25 to 1.75 wt %, and in particular 5 to 15 wt % of anionic surfactant(s), particularly preferably fatty alcohol ether sulfates of formula

H₃C₄CH₂)ₙ—(OCH₂CH₂)ₖ—OSO₃⁻M⁺,

in which n stands for values from 5 to 21, preferably from 7 to 19, particularly preferably from 9 to 17, and in particular from 11 to 13, and k stands for values of 1, 2, 3, 4, 5, 6, 7, 8, 9 or 10, preferably for 1, 2 or 3, and in particular for 2, and M stands for a cation from the group Na⁺, K⁺, NH4⁺, ½ Mg²⁺, ½Zn²⁺, preferably Na⁺.

Surface-active compounds that carry at least one quaternary ammonium group and at least one —COO⁽⁻⁾ or —SO₃⁽⁻⁾ group in the molecule are designated as zwitterionic surfactants and molecules. Particularly suitable zwitterionic surfactants and emulsifiers are the so-called betaines, such as N-alkyl-N,N-dimethyl ammonium glycinates, for example coco-alkyl dimethyl ammonium glycinate, N-acyl-aminopropyl-N,N-dimethyl ammonium glycinates, for example coco-acyl aminopropyl dimethyl ammonium glycinate, and 2-alkyl-3-carboxymethyl-3-hydroxyethyl-imidazolines, each having 8 to 18 C atoms in the alkyl or acyl group, as well as coco-acyl aminoethyl hydroxyl ethyl carboxymethyl glycinate. A preferred zwitterionic surfactant is the fatty acid amide derivative known under the INCI name cocamidopropyl betaine.

Ampholytic surfactants and emulsifiers are understood to be surface-active compounds which, in addition to a C₈-C₂₄ alkyl or acyl group, also include at least one free amino group and at least one —COOH or —SO₃H group and are capable of forming inner salts. Examples of suitable ampholytic surfactants are N-alkyl glycines, N-alkylaminopropionic acids, N-alkyl aminobutyric acids, N-alkyliminodipropionic acids, N-hydroxyethyl-N-alkylamido propyl glycines, N-alkyltaurines N-alkylsarcosines, 2-alkylaminopropionic acids and alkyl aminoacetic acids each having approximately 8 to 24 C atoms in the alkyl group. Particularly preferred ampholytic surfactants are N-coco-alkyl aminopropionate, coco-acyl aminoethyl aminopropionate and C₁₂-C₁₈ acyl sarcosine.

Particularly preferred cosmetic agents according to the invention are characterized in that they include amphoteric surfactant (s) from the groups N-alkyl glycines, N-alkyl propionic acids, N-alkyl aminobutyric acids, N-alkylimino dipropionic acids, N-hydroxyethyl-N-alkylamidopropyl glycines, N-alkyltaurines, N-alkylsarcosines, 2-alkyl aminopropionic acids each having approximately 8 to 24 C atoms in the alkyl group, alkyl aminoacetic acids each having around 8 to 24 C atoms in the alkyl group, N coco-alkyl aminopropionate, coco-acyl aminoethyl aminopropionate, C₁₂-C₁₈ acyl sarcosine, N-alkyl-N,N-dimethyl ammonium glycinates, for example coco-alkyl dimethyl ammonium glycinate, N-acyl aminopropyl-N,N-dimethyl ammonium glycinates, for example coco-acyl aminopropyl dimethylammonium glycinate, 2-alkyl-3-carboxymethyl-3-hydroxyethyl imidazolines each having 8 to 18 C atoms in the alkyl or acyl group, coco-acyl aminoethyl hydroxyethyl carboxymethyl glycinate, compounds known under the INCI name cocamidopropyl betaine, which include compounds known under the INCI name disodium cocoamphodiacetates, wherein preferred agents include the amphoteric surfactant(s) in amounts from 0.5 to 9 wt %, preferably from 0.75 to 8 wt %, and in particular from 1 to 7.5 wt %, in each case with respect to the total agent.

Particularly preferred cosmetic agents include, as amphoteric surfactants, betaines of formula (Bet-I)

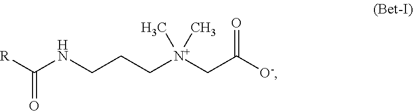

in which R stands for a straight-chained or branched, saturated or mono- or polyunsaturated alkyl or alkenyl group having 8 to 24 carbon atoms.

These surfactants are referred to in accordance with INCI nomenclature as amidopropyl betaines, wherein the representatives that derive from coconut fatty acids are preferred and are designated as coco-amido propyl betaines. Surfactants of formula (Bet-I) that are a mixture of the following representatives

H₃C—(CH₂)₇—C(O)—NH—(CH₂)₃N⁺+(CH₃)₂CH₂COO⁻

H₃C—(CH₂)₉—C(O)—NH—(CH₂)₃N⁺(CH₃)₂CH₂COO⁻

H₃C—(CH₂)ₙ—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻

are particularly preferably used in accordance with the invention.

Surfactants of formula (Bet-I) are particularly preferred used within narrower quantity ranges. Here, agents according to the invention which—with respect to their weight—include 0.25 to 8 wt %, more preferably 0.5 to 7 wt %, more preferably 0.75 to 6.5 wt %, and in particular 1 to 5.5 wt % of surfactant(s) of formula (Bet-I) are preferred.

In addition to the amphosurfactant(s) of formula (Bet-I), or alternatively thereto, the cosmetic agents according to the invention can particularly preferably include, as amphoteric surfactants, betaines of formula (Bet-II)

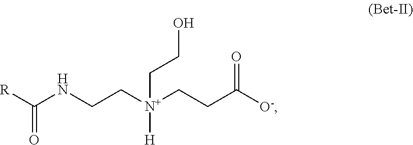

in which R stands for a straight-chained or branched, saturated or mono- or polyunsaturated alkyl or alkenyl group having 8 to 24 carbon atoms.

These surfactants are referred to in accordance with INCI nomenclature as amphoacetates, wherein the representatives that derive from coconut fatty acids are preferred and are designated as coco-amphoacetates.

For reasons related to the production process, surfactants of this type always include betaines of formula (Bet-IIa)

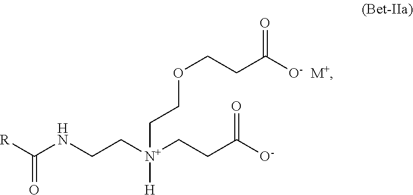

in which R stands for a straight-chained or branched, saturated or mono- or polyunsaturated alkyl or alkenyl group having 8 to 24 carbon atoms and M stands for a cation.

These surfactants are referred to in accordance with INCI nomenclature as amphodiacetates, wherein the representatives that derive from coconut fatty acids are preferred and are designated as coco-amphodiacetates.

Surfactants of formula (Bet-II) that are a mixture of the following representatives

H₃C—(CH₂)₇—C(O)—NH—(CH₂)₂NH⁺(CH₂CH₂OH)CH₂CH₂COO⁻

H₃C—(CH₂)₉—C(O)—NH—(CH₂)₂NH⁺(CH₂CH₂OH)CH₂CH₂COO⁻

H₃C—(CH₂)₁₁—C(O)—NH—(CH₂)₂NH⁺(CH₂CH₂OH)CH₂CH₂COO⁻

H₃C—(CH₂)₁₃—C(O)—NH—(CH₂)₂NH⁺(CH₂CH₂OH)CH₂CH₂COO⁻

H₃C—(CH₂)₁₅—C(O)—NH—(CH₂)₂NH⁺(CH₂CH₂OH)CH₂CH₂COO⁻

H₃C—(CH₂)₇—CH═CH—(CH₂)₇—C(O)—NH—(CH₂)₂NH⁺(CH₂CH₂OH)CH₂CH₂COO⁻

are particularly preferably used in accordance with the invention.

Surfactants of formula (Bet-II) are particularly preferred used within narrower quantity ranges. Here, agents according to the invention which—with respect to their weight —include 0.25 to 8 wt %, more preferably 0.5 to 7 wt %, more preferably 0.75 to 6.5 wt %, and in particular 1 to 5.5 wt % of surfactant(s) of formula (Bet-II) are preferred.

To summarize, cosmetic agents according to the invention in which the group R in the formulas (Bet-I) and (Bet-II) is selected from H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH—CH(CH₂)₇— or mixtures thereof are preferred.

Particularly preferred non-ionic surfactants are alkyl polyglycosides. Thus, cosmetic agents according to the invention that, as nonionic surfactants,—with respect to the weight of said agents—include 0.1 to 20 wt % alkyl polyglycosides of general formula RO—(Z)ₓ, wherein R stands for alkyl, Z for sugar and x for the number of sugar units, are particularly preferred.

Alkyl polyglycosides (APGs) are non-ionic surfactants which are produced completely from renewable raw materials (sugar building blocks, primarily glucose, for example from corn starch and fatty alcohol, for example from coconut oil). Alkyl polyglycosides are accessible by acid-catalyzed reaction (Fischer reaction) of sugars, particularly glucose (or starch), or of butyl glycosides with fatty alcohols.

Here, complex mixtures of alkylmonoglucoside (alkyl-α-D-glucopyranoside and alkyl-β-D-glucopyranoside and small amounts of alkyl-α-D-glucopyranoside and alkyl-β-D-glucofuranoside), alkyldiglucosides (-isomaltosides, -maltosides, etc.) and alkyloligoglucosides (-maltotriosides, -tetraosides, etc.) are produced. The average degree of polymerization of commercial products of which the alkyl radicals are in the range C8-C16 is 1.2-1.5.

Alkyl polyglycosides corresponding to the general formula RO—(Z)ₓ, wherein R stands for alkyl, Z for sugar and x for the number of sugar units, are preferred in accordance with the invention.

Alkyl polyglycosides in which R consists
substantially of C₈ and C₁₀ alkyl groups,
substantially of C₁₂ and C₁₄ alkyl groups,
substantially of C₈ to C₁₆ alkyl groups, or
substantially of C₁₂ to C₁₆ alkyl groups, or
substantially of C₁₆ to C₁₈ alkyl groups
   are particularly preferred.

Any mono- or oligosaccharides can be used as sugar building block Z. Usually, sugars with 5 or 6 carbon atoms and the corresponding oligosaccharides are used. Such sugars are, for example, glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

The alkyl polyglycosides useable in accordance with the invention include an average of 1.1 to 5 sugar units. Alkyl polyglycosides having x values from 1.1 to 2.0 are preferred. Alkyl glycosides in which x is 1.1 to 1.8 are most preferred.

Other surfactants which—in particular in mixture with alkylpolyglycosides—can be used particularly advantageously in the agents according to the invention are glutamates, asparaginates and sulfoacetates. Here, cosmetic agents according to the invention which—with respect to their weight—include 0.1 to 20 wt % fatty acid glutamates (acylglutamates) and/or fatty acid asparaginates (acylasparaginates) and/or alkylsulfoacetates (sulfoacetic alkyl esters) are preferred.

Acylglutamates can be described by the formula

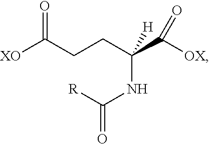

in which R—CO stands for a linear or branched acyl group having 6 to 22 carbon atoms and 0 and/or 1, 2 or 3 double bonds, and X stands for hydrogen, an alkali and/or alkaline earth metal, ammonium, alkylammonium, alkanolammonium or glucammonium.

It has surprisingly been found that mixtures of alkyl glucosides with acylglutamates have very good dermatological compatibility and an improved foaming power, both in terms of the basic foam and in terms of foam stability in the presence of water hardness.

Acylgutamates constitute known anionic surfactants that, for example, can be obtained by Schotten-Baumann acylation of glutamic acid with fatty acids, fatty acid esters or fatty acid chlorides. Commercial products are obtainable for example from Hoechst AG, Frankfurt/DE or from Ajinomoto Co. Inc., Tokyo/JP.

Typical examples of suitable acylglutamates are anionic surfactants that derive from fatty acids having 6 to 22, preferably 12 to 18 carbon atoms, such as C12/14 or C12/18 coconut fatty acid, lauric acid, myristic acid, palmitic acid and/or stearic acid. Sodium-N-cocoyl glutamate and sodium-N-stearoyl-L glutamate are particularly preferred.

The agents according to the invention can include the alkyl oligoglucosides and/or alkenyl oligoglucosides and the acylglutamates in a ratio by weight of 1:99 to 99:1, preferably 10:90 to 90:10 and in particular 80:20 to 50:50.

Acyl asparaginates can be described by the formula

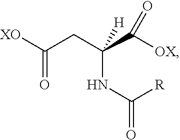

in which R—CO stands for a linear or branched acyl group having 6 to 22 carbon atoms and 0 and/or 1, 2 or 3 double bonds, and X stands for hydrogen, an alkali and/or alkaline earth metal, ammonium, alkylammonium, alkanolammonium or glucammonium.

Acyl asparaginates constitute known anionic surfactants that, for example, can be obtained by Schotten-Baumann acylation of aspartic acid with fatty acids, fatty acid esters or fatty acid chlorides. Commercial products are obtainable for example from Hoechst AG, Frankfurt/DE or from Ajinomoto Co. Inc., Tokyo/JP.

Typical examples of suitable acyl asparaginates are anionic surfactants that derive from fatty acids having 6 to 22, preferably 12 to 18 carbon atoms, such as C12/14 or C12/18 coconut fatty acid, lauric acid, myristic acid, palmitic acid and/or stearic acid. Sodium-N-cocoyl asparaginate and sodium-N-stearoyl-L asparaginate are particularly preferred.

The agents according to the invention can include the alkyl oligoglucosides and/or alkenyl oligoglucosides and the acyl asparaginates likewise in a ratio by weight of 1:99 to 99:1, preferably 10:90 to 90:10 and in particular 80:20 to 50:50.

Sulfoacetates (sulfoacetic esters) are usually salts of esters of sulfoacetic acid and can be described by the general formula

R—O—C(O)CH₂—SO₂—OX,

in which R stands for a linear or branched alkyl or alkenyl group having 6 to 22 carbon atoms and 0 and/or 1, 2 or 3 double bonds and X stands for hydrogen, an alkali and/or alkaline earth metal, ammonium, alkylammonium, alkanolammonium or glucammonium.

The use of sodium salt of sulfoacetic acid having the INCI name: sodium lauryl sulfoacetate is particularly preferred:

H₃C—(CH₂)₁₁—O—CO—CH₂—SO₂—ONa

Sodium lauryl sulfoacetate is a white, free flowing powder which reacts in a neutral manner, with good foaming power, wetting power and dispersing power.

Cationic surfactants of the quaternary ammonium compound, esterquat and amidoamine type can be used in accordance with the invention. Preferred quaternary ammonium compounds are ammonium halides, in particular chlorides and bromides, such as alkyl trimethylammonium chlorides, dialkyldimethylammonium chlorides and trialkylmethylammonium chlorides. The long alkyl chains of these surfactants preferably have 10 to 18 carbon atoms, such as in cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Other preferred cationic surfactants are imidazolium compounds known under the INCI names quaternium-27 and quaternium-83.

Particularly preferred hair treatment agents according to the invention are characterized in that they, as cationic care substance—with respect to their weight—include 0.05 to 7.5 wt %, preferably 0.1 to 5 wt %, particularly preferably from 0.2 to 3.5 wt %, and in particular 0.25 to 2.5 wt % cationic surfactant(s) from the group of quaternary ammonium compounds and/or esterquats and/or amidoamines, wherein preferred cationic surfactant(s) is/are selected from alkyltrimethylammonium chlorides having preferably 10 to 18 carbon atoms in the alkyl radical and/or dialkyldimethylammonium chlorides having preferably 10 to 18 carbon atoms in the alkyl group and/or trialkyl methyl ammonium chlorides having preferably 10 to 18 carbon atoms in the alkyl radical and/or cetyltrimethylammonium chloride and/or stearyltrimethylammonium chloride and/or distearyl dimethyl ammonium chloride and/or lauryldimethyl ammonium chloride and/or lauryldimethylbenzylammonium chloride and/or tricetylmethylammonium chloride quaternium-27 and/or quaternium-83 and/or N-methyl-N(2-hydroxyethyl)-N,N-(ditalgacyloxyethyl) ammonium methosulfate and/or N-methyl-N(2-hydroxyethyl)-N,N-(distearoyloxyethyl) ammonium methosulfate and/or N,N-dimethyl-N,N-distearoyloxyethyl ammonium chloride and/or N,N-Di-(2-hydroxyethyl)-N,N-(fatty acid ester ethyl) ammonium chloride. The care effects of the agents according to the invention can be intensified even more by using certain care substances. These are preferably selected from certain groups of care substances known per se, since these care substances harmonize outstandingly in terms of formulation and care effect with the combination used in accordance with the invention.

Hair treatment agents that are preferred in accordance with the invention are characterized in that they additionally include care substance(s)—with respect to the weight of said hair treatment agents—in amounts from 0.001 to 10 wt %, preferably 0.005 to 7.5 wt %, particularly preferably 0.01 to 5 wt %, and in particular 0.05 to 2.5 wt %, wherein preferred care substance(s) is/are selected from the group
i. L-carnitine and/or salts thereof;
ii. panthenol and/or pantothenic acid;
iii. the 2-furanones and/or derivatives thereof, in particular pantolactone;
iv. taurine and/or salts thereof;
v. niacinamide;
vi. ubiquinone
vii. ectoin;
viii. allantoin.

In the hair treatment agents according to the invention of this embodiment, the silicones are combined with at least one care substance, selected from L-carnitine and/or salts thereof, panthenol and/or pantothenic acid, 2-furanones and/or derivatives thereof, in particular pantolactone, taurine and/or salts thereof, niacinamide, ubiquinones, ectoin, and allantoin. These care substances are described below.

L-carnitine addition compounds and double salts can form as betaine. L-carnitine derivatives that are preferred in accordance with the invention are selected in particular from acetyl L-carnitine, L-carnitine fumarate, L-carnitine citrate, lauroyl-L-carnitine and particularly preferably L-carnitine tartrate. Said L-carnitine compounds are obtainable for example from the company Lonza GmbH (Wuppertal, Germany).

Hair treatment agents that are preferred in accordance with the invention are characterized in that they include—with respect to their weight—0.001 to 10 wt %, preferably 0.005 to 7.5 wt %, particularly preferably 0.01 to 5 wt %, and in particular 0.05 to 2.5 wt % of L-carnitine or L-carnitine derivatives, wherein preferred L-carnitine derivatives are selected from acetyl L-carnitine, L-carnitine fumarate, L-carnitine citrate, lauroyl-L-carnitine and in particular L-carnitine tartrate.

Panthenol (IUPAC name: (+)-(R)-2,4-dihydroxy-N-(3-hydroxypropyl)-3,3-dimethylbutyramide) is converted in the body to pantothenic acid. Pantothenic acid is a vitamin from the group of B vitamins (vitamin B5).

Hair treatment agents that are preferred in accordance with the invention are characterized in that they include—with respect to their weight—0.01 to 5 wt %, preferably 0.05 to 2.5 wt %, particularly preferably 0.1 to 1.5 wt %, and in particular 0.25 to 1 wt % panthenol ((±)-2,4-dihydroxy-N-(3-hydroxypropyl)-3,3-dimethyl-butyramide).

In addition to the care substances, the agents according to the invention can include further care substances. Their presence is not essential for achieving the effects according to the invention, but further effects, such as a pleasant feel or a pleasant sensation upon application, can result from the use of these care substances.

As a further ingredient, the agents according to the invention can particularly preferably include one or more amino acids. Amino acids that can be used particularly preferably in accordance with the invention originate from the group glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4-dihydroxy-L-phenylalanine (L-Dopa), 5′ hydroxy-L-tryptophan, L-homocysteine, S-methyl-L-methionine, S-allyl-L-cysteine sulfoxide (L-Alliin), L-trans-4-hydroxyproline, L-5-oxoproline (L-pyroglutamic acid), L-phosphoserine, creatine, 3-methyl-L-histidine, and L-ornithine, wherein both the individual amino acids as well as mixtures can be used.

Preferred agents according to the invention include one or more amino acids in narrower quantity ranges. Here, cosmetic agents that are preferred in accordance with the invention are characterized in that they include as care substance—with respect to the weight of said agents—0.01 to 5 wt %, preferably 0.02 to 2.5 wt %, particularly preferably 0.05 to 1.5 wt %, more preferably 0.075 to 1 wt %, and in particular 0.1 to 0.25 wt % amino acid(s), preferably from the group of glycine and/or alanine and/or valine and/or lysine and/or leucine and/or threonine.

As a further constituent, the agents according to the invention can include at least one carbohydrate from the group of monosaccharides, disaccharides and/or oligosaccharides. Here, cosmetic agents that are preferred in accordance with the invention are characterized in that they include, as care substance—with respect to their weight—0.01 to 5 wt %, preferably 0.05 to 4.5 wt %, more preferably 0.1 to 4 wt %, more preferably 0.5 to 3.5 wt % and in particular 0.75 to 2.5 wt % carbohydrate(s), selected from monosaccharides, disaccharides and/or oligosaccharides.

Particularly preferred agents according to the invention include, with respect to their weight,
0.005 to 0.015 wt % caffeine and 0.75 to 1.5 wt % glucose monohydrate
0.005 to 0.015 wt % caffeine and 0.75 to 1.5 wt % sucrose,
0.005 to 0.015 wt % caffeine and 0.75 to 1.5 wt % fructose.

Particularly preferred agents according to the invention include, with respect to their weight
0.005 to 0.015 wt % caffeine and 0.75 to 1.5 wt % glucose monohydrate and 0.1 to 0.25 wt % glycine,
0.005 to 0.015 wt % caffeine and 0.75 to 1.5 wt % sucrose and 0.1 to 0.25 wt % glycine,
0.005 to 0.015 wt % caffeine and 0.75 to 1.5 wt % fructose and 0.1 to 0.25 wt % glycine,
0.005 to 0.015 wt % caffeine and 0.75 to 1.5 wt % glucose monohydrate and 0.1 to 0.25 wt % alanine,
0.005 to 0.015 wt % caffeine and 0.75 to 1.5 wt % sucrose and 0.1 to 0.25 wt % alanine,
0.005 to 0.015 wt % caffeine and 0.75 to 1.5 wt % fructose and 0.1 to 0.25 wt % alanine,
0.005 to 0.015 wt % caffeine and 0.75 to 1.5 wt % glucose monohydrate and 0.1 to 0.25 wt % valine,
0.005 to 0.015 wt % caffeine and 0.75 to 1.5 wt % sucrose and 0.1 to 0.25 wt % valine,
0.005 to 0.015 wt % caffeine and 0.75 to 1.5 wt % fructose and 0.1 to 0.25 wt % valine.

The silicones used in accordance with the invention comprising structural units of formula (I) can of course be used together with other conventional silicones.

Preferred agents according to the invention are characterized in that they include at least one additional silicone.

Agents that are particularly preferred in accordance with the invention include the silicone and the other silicone(s) preferably in amounts of from 0.1 to 10 wt %, preferably from 0.25 to 7 wt % and in particular from 0.5 to 5 wt %, in each case with respect to the total agent.

Preferred silicones are described below.

Particularly preferred agents according to the invention are characterized in that they include at least one silicone of formula Si-I

(CH₃)₃Si—[O—Si(CH₃)₂]ₓ—O—Si(CH₃)₃ (Si-I),

in which x is a number from 0 to 100, preferably from 0 to 50, more preferably from 0 to 20 and particularly 0 to 10.

These silicones are referred to according to the INCI nomenclature as DIMETHICONES. Within the scope of the present invention, the compounds:
(CH₃)₃Si—O—Si(CH₃)₃ (CH₃)₃Si—[O—(CH₃)₂Si]₂—O—Si(CH₃)₃ (CH₃)₃Si—O—(CH₃)₂Si—O—Si(CH₃)₃ (CH₃)₃Si—[O—(CH₃)₂Si]₃—O—Si(CH₃)₃ (CH₃)₃Si—[O—(CH₃)₂Si]₄—O—Si(CH₃)₃ (CH₃)₃Si—[O—(CH₃)₂Si]₁₃—−O—Si(CH₃)₃ (CH₃)₃Si—[O—(CH₃)₂Si]₅—O—Si(CH₃)₃ (CH₃)₃Si—[O—(CH₃)₂Si]₁₄—O—Si(CH₃)₃ (CH₃)₃Si—[O—(CH₃)₂Si]₆—O—Si(CH₃)₃ (CH₃)₃Si—[O—(CH₃)₂Si]₁₅—O—Si(CH₃)₃ (CH₃)₃Si—[O—(CH₃)₂Si]₇—O—Si(CH₃)₃ (CH₃)₃Si—[O—(CH₃)₂Si]₁₆—O—Si(CH₃)₃ (CH₃)₃Si—[O—(CH₃)₂Si]₈—O—Si(CH₃)₃ (CH₃)₃Si—[O—(CH₃)₂Si]₁₇—O—S(CH₃)₃ (CH₃)₃Si—[O—(CH₃)₂Si]₉—O—Si(CH₃)₃ (CH₃)₃Si—[O—(CH₃)₂Si]₁₈—O—Si(CH₃)₃ (CH₃)₃Si—[O—(CH₃)₂Si]₁₀—O—Si(CH₃)₃ (CH₃)₃Si—[O—(CH₃)₂Si]₁₉—O—Si(CH₃)₃ (CH₃)₃Si—[O—(CH₃)₂Si]₁₁—O—Si(CH₃)₃ (CH₃)₃Si—[O—(CH₃)₂Si]₂₀—O—Si(CH₃)₃ (CH₃)₃Si—[O—(CH₃)₂Si]₁₂—O—Si(CH₃)₃,
   wherein (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ are particularly preferred as silicones of formula Si—I.

Of course, mixtures of the above-mentioned silicones can also be included in the agents according to the invention.

Preferred silicones that can be used in accordance with the invention have viscosities at 20° C. from 0.2 to 2 mm²s⁻¹, wherein silicones with viscosities from 0.5 to 1 mm²S⁻¹ are particularly preferred.

Particularly preferred agents according to the invention include one or more amino-functional silicones.

Preferred agents according to the invention are characterized in that they include an amino-functional silicone of formula (Si-II)

R′ₐG₃₋ₐ-Si(OSiG₂)ₙ-(OSiG_{b}R′_{2-b})ₘ—O—SiG₃₋ₐ-R′ₐ (Si-II),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, in particular 0;
b stands for a number between 0 and 1, in particular 1,
m and n are numbers of which the sum (m+n) is between 1 and 2000, preferably between 50 and 150, wherein n preferably assumes values from 0 to 1999 and in particular from 49 to 149, and m preferably assumes values from 1 to 2000, in particular from 1 to 10,
R′ is a monovalent group selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁻H₂(R″)A⁻
   -Q-N(R″)—CH₂—CH₂—N⁺R″H₂A⁻,
wherein each Q stands for a chemical bond, —CH₂—, —CH₂—CH₂—, —CH₂CH₂CH₂—, —C(CH₃)₂—, —CH₂CH₂CH₂CH₂—, —CH₂(C(CH₃)₂—, —CH(CH₃)CH₂CH₂—,
R″ stands for the same or different groups from the group —H, phenyl, benzyl, —CH₂—CH(CH₃)Ph, C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion, preferably selected from chloride, bromide, iodide or methosulfate.

Particularly preferred agents according to the invention are characterized in that they include at least one amino-functional silicone of formula (Si-IIa)

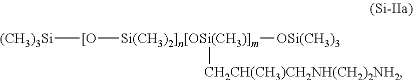

in which m and n are numbers of which the sum (m+n) is between 1 and 2000, preferably between 50 and 150, wherein n preferably assumes values from 0 to 1999 and in particular from 49 to 149, and m preferably assumes values from 1 to 2000, in particular from 1 to 10.

These silicones are referred to in accordance with the INCI declaration as trimethylsilylamidodimethicones.

Agents according to the invention that include an amino-functional silicone of formula (Si-IIb)

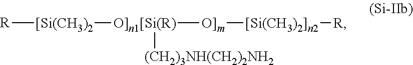

in which R stands for —OH, —O—CH₃ or a —CH₃ group and m, n1 and n2 are numbers of which the sum (m+n1+n2) is between 1 and 2000, preferably between 50 and 150, wherein the sum (n1+n2) preferably assumes values from 0 to 1999 and in particular from 49 to 149, and m preferably assumes values from 1 to 2000, in particular from 1 to 10, are particularly preferred.

These silicones are referred to in accordance with the INCI declaration as amidodimethicones.

Regardless of which amino-functional silicones are used, agents according to the invention which include an amino-functional silicone of which the amine number is above 0.25 meq/g, preferably above 0.3 meq/g and in particular above 0.4 meq/g are preferred. The amine number stands here for milliequivalents of amine per gram of amino-functional silicone. It can be determined by titration and expressed in the unit mg KOH/g.

Agents that are preferred in accordance with the example are characterized in that they include, with respect to their weight, 0.01 to 10 wt %, preferably 0.1 to 8 wt %, particularly preferably 0.25 to 7.5 wt %, and in particular 0.5 to 5 wt % of amino-functional silicone(s).

The silicones described above have a backbone, which is composed of —Si—O—Si units. Of course, these Si—O—Si units can also be interrupted by carbon chains. Corresponding molecules are accessible by chain extension reactions and are preferably used in the form of silicone-in-water emulsions.

Agents that are likewise preferred in accordance with the invention are characterized in that they include at least one silicone of formula Si-IV

R₃Si—[O—SiR₂]ₓ—(CH₂)ₙ—[O—SiR₂]_{y}—O—SiR₃ (Si-IV),

in which R stands for the same or different groups from the group —H, phenyl, benzyl, —CH₂—CH(CH₃)Ph, C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y stand for a number from 0 to 200, preferably from 0 to 10, more preferably from 0 to 7, and in particular 0, 1, 2, 3, 4, 5 or 6, and n stands for a number from 0 to 10, preferably from 1 to 8, and in particular for 2, 3, 4, 5 or 6.

The silicones are preferably water-soluble. Agents that are preferred in accordance with the invention are characterized in that they include at least one water-soluble silicone.

It has been found that certain proteolipids enhance the effect of the combination according to the invention even further and improve the prevention of damage to the hair structure by UV radiation even further. As a further ingredient, the agents according to the invention can include at least one proteolipid of formula (P-1)

R′—X—R″ (P-I)

in which
R′ stands for a straight-chained or branched, saturated or unsaturated hydrocarbon having 11 to 24 carbon atoms,
R″ is a protein, a peptide or a protein hydrolyzate.
X stands for —C(O)O— or —N⁺(R^{III}₂)R^{IV}— or N(R^{III})R^{IV}— or —C(O)—N(R^{V})R^{VI}—,
R^{III} is —(CH₂)ₓ—CH₃ with x=0-22, and
R^{IV} is —CH₂—CH(OH)—CH₂— or —(CH₂)ₓ— with x=0-22;
R^{V} and R^{V}, independently of one another, stand for —H or —(CH₂)ₓ—CH₃ with x=0-22;
   provided that R″ stands for keratin or a keratin hydrolyzate when X stands for —C(O)O—.

The proteolipids are preferably used within certain amounts in the agents according to the invention. Preferred cosmetic agents according to the invention include—with respect to their weight—0.01 to 10 wt %, preferably 0.02 to 5 wt %, particularly preferably 0.05 to 2.5 wt %, more preferably 0.1 to 1 wt %, and in particular 0.15 to 0.5 wt % proteolipid(s).

The group R″ in formula (P-1) stands for a peptide or a protein or a protein hydrolyzate. If X=—C(O)O—, R″ is selected from the group of keratin or keratin hydrolyzate.

Preferred groups R″ are oligopeptides which comprise at least one amino acid sequence Glu-Glu-Glu

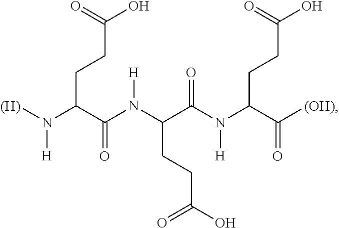

wherein the amino group can be present in free or protonated form and the carboxy groups can be present in free or deprotonated form.

In this, as in all formulas below, the bracketed hydrogen atom of the amino group as well as the bracketed hydroxyl group of the acid function means that the relevant groups may be present as such (it is then an oligopeptide with the relevant number of amino acids as shown (in the above formula 3)), or that the amino acid sequence is present in an oligopeptide which comprises further amino acids—depending on where the further amino acid(s) is/are bonded, the bracketed constituents of the above-mentioned formula are replaced by the further amino acid group(s).

Oligopeptides within the meaning of the present application are condensation products of amino acids linked in an acid amide-like manner by peptide bonds, comprising at least 3 and at most 25 amino acids,

In hair treatment agents that are preferred in accordance with the invention corresponding to the above described embodiment the oligopeptide (=the group R″) comprises 5 to 15 amino acids, preferably 6 to 13 amino acids, particularly preferably 7 to 12 amino acids, and in particular 8, 9 or 10 amino acids.

Depending on whether further amino acids are bonded to the sequence Glu-Glu-Glu and depending on the nature of these amino acids, as well as in dependence of the selection of the groups R′ and, as appropriate, R^{III} and R^{IV}, the molar mass of the proteolipid included in the agents according to the invention can vary. Cosmetic agents that are preferred in accordance with the invention are characterized in that the proteolipid has a molar mass from 1000 to 30,000 Da, preferably from 1250 to 25,000 Da, particularly preferably 1500 to 20,000 Da and in particular from 2000 to 15,000 Da.

Oligopeptides which not only consist of the three glutamic acids, but have other amino acids bonded to this sequence are preferably used as group R″. These further amino acids are preferably selected from specific amino acids, whereas certain other representatives are less preferred in accordance with the invention.

It is thus preferred if the group R″ of the proteolipids used in the agents according to the invention does not include methionine. It is further preferred if the group R″ of proteolipids used in the agents according to the invention does not include cysteine and/or cystine.

It is more preferred when the group R″ of proteolipids used in the agents according to the invention does not include aspartic acid and/or asparagine. It is more preferred if the group R″ of proteolipids used in the agents according to the invention does not include serine and/or threonine.

By contrast, it is preferred if the group R″ of proteolipids used in the agents according to the invention includes tyrosine. It is more preferred if the group R″ of proteolipids used in the agents according to the invention includes leucine. It is more preferred if the group R″ of proteolipids used in the agents according to the invention includes isoleucine. It is more preferred if the group R″ of proteolipids used in the agents according to the invention includes arginine. It is more preferred if the group R″ of proteolipids used in the agents according to the invention includes valine.

Oligopeptides that are particularly preferred as the group R″ and amino acid sequences included in the preferred oligopeptides will be described below:

A particularly preferred oligopeptide additionally includes tyrosine, which is preferably bonded via its acid function to the Glu-Glu-Glu-sequence. Cosmetic agents that are preferred in accordance with the invention are therefore characterized in that the oligopeptide included as group R″ in the proteolipids of formula (I) has at least one amino acid sequence Tyr-Glu-Glu-Glu, wherein the amino group can be present in free or protonated form and the carboxy groups can be present in free or deprotonated form.

A further particularly preferred oligopeptide additionally includes isoleucine, which is preferably bonded via its amino function to the Glu-Glu-Glu-sequence. Cosmetic agents that are preferred in accordance with the invention are therefore characterized in that the oligopeptide included as group R″ in the proteolipids of formula (I) has at least one amino acid sequence Glu-Glu-Glu-Ile, wherein the amino group can be present in free or protonated form and the carboxy groups can be present in free or deprotonated form.

Oligopeptides which have both of the aforementioned amino acids (tyrosine and isoleucine) are preferred in accordance with the invention. Hair treatment agents according to the invention that are particularly preferred here are those in which the oligopeptide included as group R″ in the proteolipids of formula (I) has at least one amino acid sequence Tyr-Glu-Glu-Glu-Ile, wherein the amino group can be present in free or protonated form and the carboxy groups can be present in free or deprotonated form.

More preferred oligopeptides additionally include arginine, which is preferably bonded to isoleucine. Cosmetic agents that are preferred in accordance with the invention are therefore characterized in that the oligopeptide included as group R″ in the proteolipids of formula (I) has least one amino acid sequence Tyr Glu-Glu-Glu-Ile-Arg, wherein the amino groups can be present in free or protonated form and the carboxy groups can be present in free or deprotonated form.

Even more preferred oligopeptides additionally include valine, which is preferably bonded to the arginine. Cosmetic agents that are more preferred in accordance with the invention are therefore characterized in that the oligopeptide included as group R″ in the proteolipids of formula (I) has at least an amino acid sequence Tyr-Glu-Glu-Glu-Ile-Arg-Val, wherein the amino groups can be present in free or protonated form and the carboxy groups can be present in free or deprotonated form.

Even more preferred oligopeptides additionally include leucine, which is preferably bonded to valine. Cosmetic agents that are more preferred in accordance with the invention are characterized in that the oligopeptide included as group R″ in the proteolipids of formula (I) has at least one amino acid sequence Tyr-Glu-Glu-Glu-Ile-Arg-Val-Leu, wherein the amino groups can be present in free or protonated form and the carboxy groups can be present in free or deprotonated form.

Particularly preferred oligopeptides additionally include leucine, which is preferably bonded to the tyrosine. Cosmetic agents that are more preferred in accordance with the invention are characterized in that the oligopeptide included as group R″ in the proteolipids of formula (I) has at least one amino acid sequence Leu-Tyr-Glu-Glu-Glu-Ile-Arg-Val-Leu, wherein the amino groups can be present in free or protonated form and the carboxy groups can be present in free or deprotonated form.

In summary, cosmetic agents that are preferred in accordance with the invention are in particular those which include at least one proteolipid of formula (I), in which R″ has at least one amino acid sequence Leu-Tyr-Glu-Glu-Glu-Ile-Arg-Val-Leu, wherein the amino groups can be present in free or protonated form and the carboxy groups can be present in free or deprotonated form.

As already mentioned, R″ is selected from the group of keratin or keratin hydrolyzate, when X=—C(O)O—.

In all other cases, the group R″ in formula (P-I) can stand for a peptide or a protein or a protein hydrolyzate, wherein protein hydrolyzates are preferred. Protein hydrolyzates are product mixtures obtained by acid-, base- or enzyme-catalyzed degradation of proteins. Protein hydrolyzates of both plant and animal origin can be used in accordance with the invention.

Animal protein hydrolyzates are, for example, elastin, collagen, keratin, silk and milk protein hydrolyzates, which can also be present in the form of salts. Such products are marketed for example under the trade names Dehylan® (Cognis), Promois® (Interorgana) Collapuron® (Cognis), Nutrilan® (Cognis), Gelita-Sol® (German gelatin factories Stoess & Co), Lexein® (Inolex) and Kerasol® (Croda).

The use of protein hydrolyzates of plant origin, for example soy, almond, rice, pea, potato and wheat protein hydrolyzates, is preferred in accordance with the invention. Such products are obtainable, for example, under the trade names Gluadin® (Cognis), DiaMin® (Diamalt), Lexenin® (Inolex) and Crotein® (Croda).

Regardless of the choice of X in formula (P-1), the group R″ is preferably selected from keratin or keratin hydrolyzates. Preferred cosmetic agents according to the invention are characterized in that they include at least one proteolipid of formula (P-1), in which R″ stands for keratin or a keratin hydrolyzate.

In particular, preferred cosmetic agents according to the invention are those which include at least one proteolipid of formula (P-1), in which R^{III} means —CH₃ and R^{IV} stands for —(CH₂)ₓ— with x=0, 1, 2, 3, 4, 5, 6, 7 or 8.

Furthermore, particularly preferred cosmetic agents according to the invention are characterized in that they include at least one proteolipid of formula (I), in which X is —N⁺(CH₃)₂—CH₂—CH(OH)—CH₂— and R′ stands for —(CH₂)₁₇—CH₃.

Cosmetic agents according to the invention that are more preferred are likewise characterized in that they include at least one proteolipid of formula (P-1), in which X stands for —C(O)—O— and R′ stands for —(CH₂)₁₇—CH₃.

It has proven to be advantageous if protein hydrolyzates are used in addition to the proteolipids. These protein hydrolyzates intensify the effect of the proteolipids and are in turn intensified in terms of their own effects. The protein hydrolyzates have been described in greater detail above as the group R″.

To summarize, preferred cosmetic agents according to the invention are those which additionally—with respect to their weight—include 0.01 to 10 wt %, preferably 0.05 to 7 wt %, particularly preferably 0.1 to 5 wt %, more preferably 0.25 to 2.5 wt %, and in particular 0.5 to 2.0 wt % of protein hydrolyzate(s), preferably keratin hydrolyzate(s).

For aesthetic reasons “clear” products are often preferred by consumers. Cosmetic agents that are preferred in accordance with the invention are therefore characterized in that they are transparent or translucent.

The term ‘transparent’ or ‘translucent’ is understood within the scope of the present invention to mean a composition that has an NTU value of less than 100. The NTU value (Nephelometric Turbidity Unit; NTU) is a unit used in water treatment for turbidity measurements in liquids. It is the unit of the turbidity of a liquid measured using a calibrated nephelometer.

A further subject matter of the present invention is the use of silicones that have groups of formula (I)

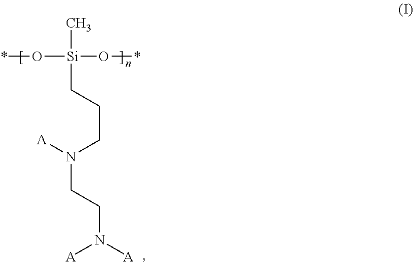

in which
n stands for values from 1 to 1000,
A stands for a group

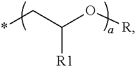

in which R1 stands for —H or —CH₃ and R stands for a straight-chained or branched alkyl group having 8 to 24 C atoms and a stands for an integer from 1 to 20,
   for reducing damage to the hair structure caused by external influences, in particular by UV radiation.

The silicones are preferably used in accordance with the invention in combination with at least one copolymer having monomer units of formulas (V) and (VI)

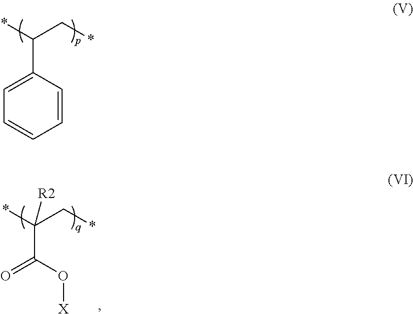

in which R2 stands for —H or —CH₃ and X stands for —H, an alkali metal cation or for a straight-chained or branched alkyl group having 1 to 24 C atoms.

That described in respect of the agents according to the invention also applies, mutatis mutandis, to further preferred embodiments of the uses according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent, including—in each case with respect to the weight of the hair treatment agent—
a) 0.1 to 20 wt % of at least one silicone, which has groups of formula (I) 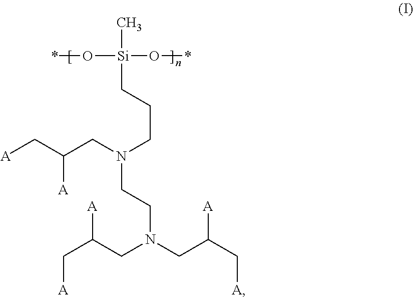
wherein
n stands for values from 1 to 1000, and
A stands for a group 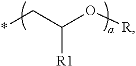
in which R1 stands for —H or —CH₃ and R stands for a straight-chained or branched alkyl group having 8 to 24 C atoms and a stands for an integer from 1 to 20;
b) 0.1 to 20 wt % of at least one copolymer, which has monomer units of formulas (V) and (VI) 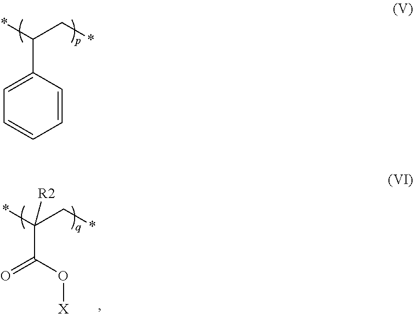
wherein R2 stands for —H or —CH₃ and X stands for —H, an alkali metal cation, or a straight-chained or branched alkyl group having 1 to 24 C atoms; and
c) 0.1 to 10 wt % of (RS)-2-cyano-3,3-diphenylacrylic acid-2-ethylhexylester.

2. The hair treatment agent according to claim 1, wherein the agent includes 0.1 to 20 wt % the at least one silicone, which has groups of formula (Ia) 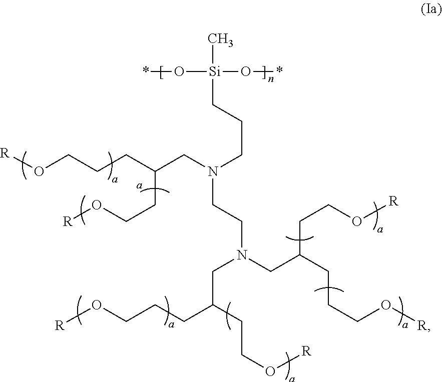
wherein
R is selected from
—(CH₂)ₖ—CH₃ with k=7, 8, 9, 10, 11, 12, 13, 14, 15, 16 or 17, and
—CH(CH₂CH₃)—(CH₂)₅, and
a stands for an integer from 1 to 20.

3. The hair treatment agent according to claim 1, wherein the agent includes 0.1 to 20 wt % the at least one silicone, which is represented by formula (II): 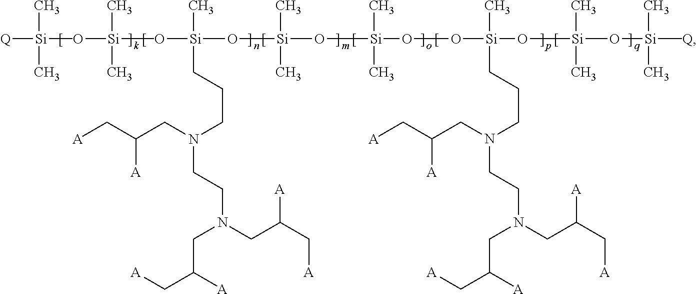
wherein
A stands for a group 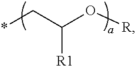
in which R1 stands for —H or —CH₃ and R stands for a straight-chained or branched alkyl group having 8 to 24 C atoms and a stands for an integer from 1 to 20;
Q stands for —OH or —CH₃ or —OCH₃; and
the indices k, n, m, o, p and q stand for integers from 0 to 1000, provided that
n+p>0 and k+m+o+q>0,
wherein the units with the indices k, n, m, o, p, q can be present in the molecule as a block or randomly distributed.

4. The hair treatment agent according to claim 1, wherein the agent includes 0.1 to 20 wt % the at least one silicone, which is represented by formula (III): 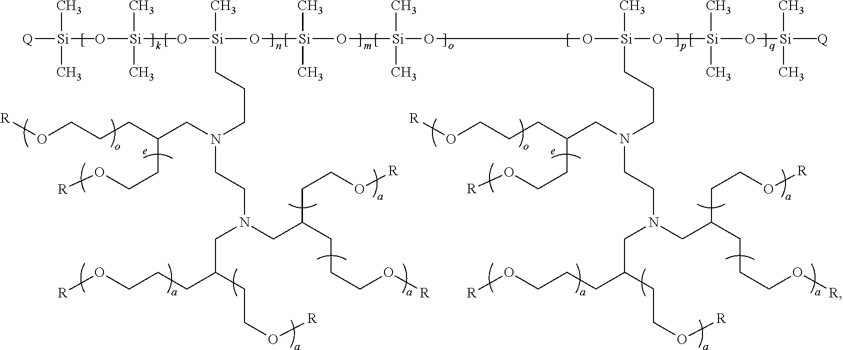
wherein
R is selected from
—(CH₂)ₖ—CH₃ with k=7, 8, 9, 10, 11, 12, 13, 14, 15, 16 or 17, and
—CH(CH₂CH₃)—(CH₂)₅;
a stands for an integer from 1 to 20;
Q stands for —OH or —CH₃ or —OCH₃; and
the indices k, n, m, o, p and q stand for integers from 0 to 1000, provided that
n+p>0 and k+m+o+q>0
wherein the units with the indices k, n, m, o, p, q can be present in the molecule as a block or randomly distributed.

5. The hair treatment agent according to claim 1, wherein the agent includes 0.1 to 20 wt % the at least one silicone, which is represented by formula (IV): 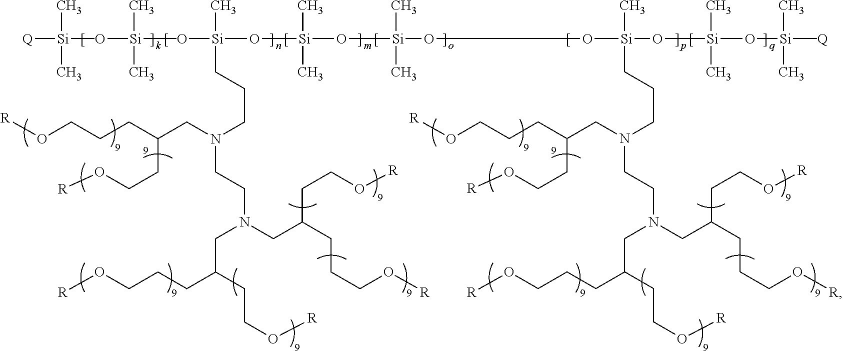
wherein
R stands for —(CH₂)₁₂—CH₃;
Q stands for —OH or —CH₃ or —OCH₃; and
the indices k, n, m, o, p and q stand for integers from 0 to 1000, provided that
n+p>0 and k+m+o+q>0
wherein the units with the indices k, n, m, o, p, q can be present in the molecule as a block or randomly distributed.

6. The hair treatment agent according to claim 1, wherein the agent includes 0.5 to 17.5 wt % the at least one copolymer, which has monomer units of formulas (V) and (VIa) 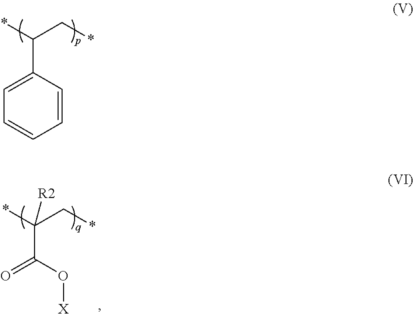
wherein X stands for H⁺ or Na⁺.

7. The hair treatment agent according to claim 1, wherein the (RS)-2-cyano-3,3-diphenylacrylic acid-2-ethylhexylester is included at a concentration of 0.25 to 9 wt %.

8. The hair treatment agent according to claim 1, wherein a ratio by weight of ingredients b) to a) lies in the range from 1:50 to 50:1.

9. The hair treatment agent according to claim 1, wherein a ratio by weight of ingredients a) to c) lies in the range from 1:50 to 50:1.

10. A method for reducing damage to the hair structure caused by external influences, in particular by UV radiation, including applying to the hair silicones which have groups of formula (I) 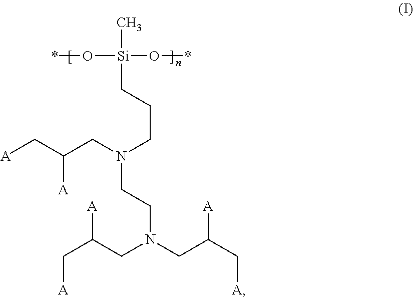
wherein
n stands for values from 1 to 1000 and
A stands for a group 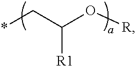
in which R1 stands for —H or —CH₃ and R stands for a straight-chained or branched alkyl group having 8 to 24 C atoms and a stands for an integer from 1 to 20.
